(19) United States
(12) Patent Application Publication
(10) Pub. No.: US 2010/0240894 A1
(43) Pub. Date: Sep. 23, 2010

(54) METHOD FOR PRODUCING AMINES FROM GLYCERIN

(75) Inventors: Martin Ernst, Heidelberg (DE); Bram Willem Hoffer, Fanwood, NJ (US); Johann-Peter Melder, Bohl-Iggelheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/675,413

(22) PCT Filed: Aug. 15, 2008
(86) PCT No.: PCT/EP08/60741

§ 371 Date: Feb. 26, 2010

(30) Foreign Application Priority Data
Aug. 29, 2007 (EP) 07115214.4

(51) Int. Cl. C07D 241/04; C07C 209/16; B01J 19/00

(52) U.S. Cl. 544/402; 564/479; 422/129

(57) ABSTRACT

The present invention relates to a process for preparing amines by reacting glycerol with hydrogen and an aminating agent from the group of ammonia and primary and secondary amines in the presence of a catalyst at a temperature of from 100° C. to 400° C. and a pressure of from 0.01 to 40 MPa (from 0.1 to 400 bar). Preference is given to using glycerol based on renewable raw materials. The catalyst preferably comprises one metal or a plurality of metals or one or more oxygen compounds of the metals of groups 8 and/or 9 and/or 10 and/or 11 of the Periodic Table of the Elements. The invention further relates to the use of the reaction products as an additive in cement or concrete production and in other fields of use. This invention further provides the compounds 1,2,3-triaminopropane, 2-aminomethyl-6-methylpiperazine, 2,5-bis(aminomethyl)piperazine and 2,6-bis(aminomethyl)piperazine.

The present invention relates to a process for preparing amines from glycerol and to the use thereof. The present invention further relates to 1,2,3-triaminopropane, 2-aminomethyl-6-methylpiperazine, 2,5-bis(aminomethyl)piperazine and 2,6-bis(aminomethyl)piperazine and to their preparation by hydrogenating amination of glycerol.

The industrial scale preparation of industrially important amino alkanols, such as ethanolamine and isopropanolamine, and their conversion products such as ethylenediamine, 1,2-propylenediamine and piperazine, generally proceeds from ethylene oxide or propylene oxide as a C₂ or C₃ synthesis unit.

For instance, ethanolamine and isopropanolamine are synthesized by reaction of ammonia with ethylene oxide and propylene oxide respectively. As further products, this reaction also affords the corresponding dialkanolamines and trialkanolamines. The ratio of monoalkanolamines to di- and trialkanolamines can be controlled through the use amounts of ammonia relative to alkylene oxide. In order to obtain a higher proportion of trialkanolamines, mono- and dialkanolamines can be recycled into the reactor.

In a further reaction stage, the monoalkanolamines thus obtained can be converted further by reaction of hydrogen and ammonia to give ethylenediamine and 1,2-propylenediamine.

1,3-Diaminopropane is obtainable on the industrial scale by reacting ammonia with acrylonitrile and subsequent hydrogenation, and acrylonitrile is generally prepared on the industrial scale by ammoxidation of the C₃ unit propene.

As an alternative raw material source to the ethene- or propene-based petrochemical feedstocks mentioned, raw materials based on renewable raw materials might gain a higher status.

In the future, growing significance might be gained by glycerol which is obtained as a by-product in fat hydrolysis and in biodiesel production.

Already commercially available glycerol-based amines are the so-called polyetheramines. The synthesis of polyetheramines by amination of polyalkylenediols or -triols is described, for example, in the review article by Fischer et al. (A. Fischer, T. Mallat, A. Baiker, Catalysis Today, 37 (1997), 167-189). Polyalkylenetriols can be obtained, for example, by reaction of ethylene oxide or propylene oxide with glycerol.

Additionally known is the synthesis of serinol (2-amino-1,3-propanediol) or serine by oxidation of glycerol with subsequent reductive amination (H. Kimura, K. Tsuto, Journal of the American Oil Chemist Society, 70 (1993), 1027-1030).

In a variant described in the aforementioned literature source, glycerol is first oxidized to 2,3-dihydroxypropionic acid (glycerol acid) and then aminated in the presence of hydrogen and ammonia over a catalyst system consisting of a mixture of carbon-supported palladium and ruthenium in a reductive manner to give DL-serine.

In a second variant, glycerol is oxidized only up to dihydroxyacetone and then, as described above, converted in a reductive amination reaction to serinol (2-amino-1,3-propanediol), which is then oxidized further to serine. According to the disclosure, under the conditions of the reductive amination over Pd or Ru catalysts, the decomposition products glycine and monoethanolamine can form from serine and serinol respectively by dehydrogenation and decarbonylation reactions.

It was an object of the present invention to utilize glycerol as a source for the preparation of amines. The intention was to provide a process which both allows important industrial amines and glycerol-based specialty amines, and also piperazine derivatives to be obtained, in order to be able to utilize the glycerol raw material in an optimal manner.

Industrial amines refer to those amines which are typically obtained on the basis of petrochemical raw materials, for example monoamines such as methylamine, ethylamine, isopropylamine or n-propylamine, diamines such as ethylenediamine, 1,2-propanediamine or 1,3-propanediamine, alkanolamines such as monoethanolamine, 2-aminopropan-1-ol or 1-aminopropan-2-ol, or piperazine.

Glycerol-based specialty amines are amines which are characterized in that at least one OH group of the glycerol has been substituted for a primary amino group, a secondary amino group or a tertiary amino group, for example 1, 2,3-triaminopropane, 1,3-diaminopropan-2-ol, 1,2-diaminopropan-3-ol, 1-aminopropanediol or 2-aminopropanediol. These compounds have a high number of functionalities and may therefore be important intermediates in the synthesis of organic compounds, such as crop protection compositions, pharmaceuticals, stabilizers, etc.

Derivatives of piperazine (piperazine derivatives) such as 2-methylpiperazine, 2,6-dimethylpiperazine, 2,5-dimethylpiperazine, 2,5-bis(aminomethyl)piperazine, 2,6-bis(aminomethyl)piperazine, 2-aminomethyl-5-methylpiperazine and 2-aminomethyl-6-methylpiperazine may likewise be important synthesis units.

The conversion of glycerol to the compounds mentioned should include only few reaction steps in order to keep the capital costs as low as possible. By virtue of easy-to-perform adjustments to the process conditions, for example pressure and temperature, reaction time, catalyst hourly space velocity, variation of the molar aminating agent to glycerol ratio, and also by virtue of the selection of the catalyst used, it should additionally be possible to regulate the composition of the reaction effluent within certain limits in order thus to be able to react better to variations in demand and sales in relation to industrial amines, glycerol-based specialty amines or piperazine derivatives.

According to the invention, a process has been found for preparing amines by reacting glycerol with hydrogen and an aminating agent from the group of ammonia and primary and secondary amines in the presence of a catalyst at a temperature of from 100° C. to 400° C. and a pressure of from 0.01 to 40 MPa (from 0.1 to 400 bar).

The conversion of glycerol in the presence of hydrogen and an aminating agent selected from the group consisting of ammonia and primary and secondary amines is referred to hereinafter as hydrogenating amination of glycerol, or else only as hydrogenating amination for short.

The reactants used in the reaction are glycerol, hydrogen and an aminating agent selected from the group consisting of ammonia and primary and secondary amines.

Glycerol is typically obtained as a by-product in the conversion of fats and oils to fatty acids (fat hydrolysis) or fatty acid methyl esters (biodiesel). The preparation of glycerol from fats and oils is described, for example, in Ullmann (Ullmann's Encyclopedia of Industrial Chemistry, Glycerol, Chapter 4.1 “Glycerol from Fat and Oils”, Wiley-VCH-Verlag, Electronic Edition, 2007).

Glycerol can also be prepared proceeding from the petrochemical starting material propene. There is likewise a review of the synthesis of glycerol from propene in Ullmann (Ullmann's Encyclopedia of Industrial Chemistry, “Glycerol”, Chapter 4.1 “Synthesis from Propene”, Wiley-VCH-Verlag, Electronic Edition, 2007). For the process according to the invention, the preparation route by which glycerol has been obtained is generally unimportant. Glycerol on a vegetable, animal or petrochemical basis is suitable as a starting material for the process according to the invention.

Particular preference is given to using glycerol based on renewable raw materials, for example glycerol which is obtained as a by-product from fat hydrolysis or biodiesel production.

Glycerol is obtainable in various qualities, for example as crude quality, as technical quality or in pharmaceutical quality.

Glycerol which is obtainable in crude quality is obtained typically in biodiesel production. For biodiesel production, vegetable oils and fats in which one glycerol molecule is esterified with in each case three fatty acid molecules are transesterified, typically after heating with addition of catalyst (NaOH or sodium methoxide) with methanol to give fatty acid methyl esters (biodiesel). The coproduct formed is glycerol. By-products are sodium salts of the fatty acids (soaps). The aqueous mixture of glycerol, soaps, methanol, catalyst and water is generally removed by physical means from the lipophilic fatty acid methyl ester. Acidification with hydrochloric acid forms fatty acids and sodium chloride. Crude glycerol and fatty acid are generally separated by phase separation. The methanol is removed by distillation.

Crude glycerol generally has a water content of from 5 to 30% by weight, typically from 10 to 15% by weight, a salt content of from 0.1 to 10% by weight, typically from 5 to 7% by weight, and a methanol content of less than 1% by weight, typically from 0.1 to 0.5% by weight.

Glycerol in technical quality or in pharmaceutical quality is generally purified by distillation in one or more stages in order to reduce the salt content and the color number.

Glycerol in technical quality is obtainable in various glycerol contents (e.g. 99.8% or glycerol according to the Nobel test with 99.5%).

In the case of glycerol in pharmaceutical quality (e.g. European Pharmacopoeia (Ph. Eur.), United States Pharmacopeia (U.S.P.), Japanese Pharmacopoeia), strict specification limits have to be complied with in relation to the content of particular secondary components and physical parameters. In the case of glycerol in pharmaceutical quality, the glycerol content is typically more than 99%, e.g. 99.5% or 99.8%.

In the process according to the invention, preference is given to using glycerol in technical quality or pharmaceutical quality with a glycerol content of at least 95%, preferably at least 98% and more preferably at least 99%.

The glycerol used is typically clear and light in color and generally has a color number of less than 100 APHA, preferably less than 50 APHA and more preferably less than 20 APHA.

The salt content of the glycerol used is typically less than 0.1% by weight, preferably less than 0.05% by weight.

The glycerol used may also comprise water, in which case the water content should generally not be more than 50% by weight, preferably less than 20% by weight and more preferably less than 5% by weight.

The glycerol may also comprise sulfur-containing components.

In general, these quality requirements with regard to water content, color number and glycerol content are met by most commercially available technical and pharmaceutical glycerol qualities.

In the case of use of crude glycerol, owing to a higher salt content, there may possibly be undesired deposits in the reactor and, owing to a higher content of by-products, stronger discoloration of the inventive amines. When crude glycerol is to be used in the process, measures must possibly be undertaken, such as more frequent cleaning of the reactor or purification of the reaction effluent in order to obtain a product suitable for the particular end use.

As a further feedstock, hydrogen is used in the process.

The aminating agent is selected from the group consisting of ammonia, primary amines and secondary amines.

As well as ammonia, it is equally possible to use primary or secondary amines as aminating agents.

For example, the following mono- and dialkylamines may be used as aminating agents: methylamine, dimethylamine, ethylamine, diethylamine, n-propylamine, di-n-propylamine, isopropylamine, diisopropylamine, isopropylethylamine, n-butylamine, di-n-butylamine, s-butylamine, di-s-butylamine, isobutylamine, n-pentylamine, s-pentyl-amine, isopentylamine, n-hexylamine, s-hexylamine, isohexylamine, cyclohexylamine, aniline, toluidine, piperidine, morpholine and pyrrolidine.

However, preference is given to using inexpensive aminating agents available on the industrial scale, such as ammonia, methylamine or diethylamine.

Particular preference is given to using ammonia as the aminating agent.

Optionally, water can be added to the process.

The catalysts used in the process according to the invention comprise one or more metals of groups 8 and/or 9 and/or 10 and/or 11 of the Periodic Table of the Elements (Periodic Table in the IUPAC version of 06.22.2007, http://www.iupac.org/reports/periodic_table/IUPAC_Periodic_Table-22Jun07b.pdf). Examples of such metals are Cu, Co, Ni and/or Fe, and also noble metals such as Rh, Ir, Ru, Pt, Pd, and Re.

The abovementioned metals may be used in the form of metal meshes or grids.

In a preferred embodiment, the metals are used in the process according to the invention in the form of Raney sponge or skeletal catalysts. Particular preference is given to using Raney nickel and/or cobalt catalysts.

Raney nickel or cobalt catalysts are prepared typically by treating an aluminum-nickel or aluminum-cobalt alloy with concentrated sodium hydroxide solution, which leaches out the aluminum and forms a metallic nickel or cobalt sponge. The preparation of Raney catalysts is described, for example, in the Handbook of Heterogeneous Catalysis (M. S. Wainright in G. Ertl, H. Knözinger, J. Weitkamp (eds.), Handbook of Heterogeneous Catalysis, Vol. 1, Wiley-VCH, Weinheim, Germany 1997, page 64 ff.). Such catalysts are obtainable, for example, as Raney® catalysts from Grace or as Sponge Metal® catalysts from Johnson Matthey.

The catalysts usable in the process according to the invention may also be prepared by reducing so-called catalyst precursors.

The catalyst precursor comprises an active composition which comprises one or more catalytically active components and optionally a support material.

The catalytically active components are oxygen compounds of the metals of groups 8 and/or 9 and/or 10 and/or 11 of the Periodic Table of the Elements (Periodic Table in the IUPAC version of 06.22.2007), for example their metal oxides or hydroxides (examples if appropriate), such as CoO, NiO, Mn₃O₄, CuO, RuO(OH)ₓ and/or mixed oxides thereof, such as LiCoO₂.

The mass of the active composition is the sum of the mass of the support material and of the mass of the catalytically active components.

The catalyst precursors used in the process may, as well as the active composition, comprise shaping media such as graphite, stearic acid, phosphoric acid or further processing assistants.

The catalyst precursors used in the process may further comprise one or more doping elements (oxidation stage 0) or inorganic or organic compounds thereof, selected from groups 1 to 14 of the Periodic Table. Examples of such elements or compounds thereof are: transition metals such as Mn or manganese oxides, Re or rhenium oxides, Cr or chromium oxides, Mo or molybdenum oxides, W or tungsten oxides, Ta or tantalum oxides, Nb or niobium oxides or niobium oxalate, V or vanadium oxides or vanadyl pyrophosphate, zinc or zinc oxides, silver or silver oxides, lanthanides such as Ce or CeO₂ or Pr or Pr₂O₃, alkali metal oxides such as K₂O, alkali metal carbonates such as Na₂CO₃ and K₂CO₃, alkaline earth metal oxides such as SrO, alkaline earth metal carbonates such as MgCO₃, CaCO₃, BaCO₃, phosphoric anhydrides and boron oxide (B₂O₃).

In the process according to the invention, the catalyst precursors are preferably used in the form of catalyst precursors which consist only of catalytically active composition, if appropriate a shaping assistant (for example graphite or stearic acid) if the catalyst is used as a shaped body and if appropriate one or more doping elements, but do not comprise any further catalytically active accompanying substances in addition. In this connection, the support material is considered to form part of the catalytically active composition.

The compositions specified below relate to the composition of the catalyst precursor after its last heat treatment, which is generally a calcination, and before its reduction with hydrogen.

The proportion of the active composition based on the total mass of the catalyst precursor is typically 70% by weight or more, preferably from 80 to 100% by weight, more preferably from 90 to 99% by weight, especially from 92 to 98% by weight.

In a preferred embodiment, the active composition of the catalyst precursor does not comprise any support material.

The active composition of catalyst precursors which do not comprise any support material preferably comprises one or more active components selected from the group consisting of CoO, NiO, Mn₃O₄, CuO, RuO(OH)), and LiCoO2.

More preferably, the active composition of catalyst precursors which do not comprise any support material comprises NiO and/or CoO.

Such catalyst precursors are, for example,

catalysts disclosed in patent application PCT/EP2007/052013 which, before reduction with hydrogen, comprise a) cobalt and b) one or more elements of the alkali metal group, of the alkaline earth metal group, of the rare earth group or zinc or mixtures thereof, where elements a) and b) are present at least partly in the form of their mixed oxides, for example LiCoO2, or
catalysts disclosed in EP-A-0636409, whose catalytically active composition, before reduction with hydrogen, comprises from 55 to 98% by weight of Co, calculated as CoO, from 0.2 to 15% by weight of phosphorus, calculated as H₃PO₄, from 0.2 to 15% by weight of manganese, calculated as MnO₂, and from 0.2 to 15% by weight of alkali metal, calculated as M₂O (M=alkali metal), or
catalysts disclosed in EP-A-0742045, whose catalytically active composition, before reduction with hydrogen, comprises from 55 to 98% by weight of Co, calculated as CoO, from 0.2 to 15% by weight of phosphorus, calculated as H₃PO₄, from 0.2 to 15% by weight of manganese, calculated as MnO₂, and from 0.05 to 5% by weight of alkali metal, calculated as M₂O (M=alkali metal).

In a further preferred embodiment, the active composition comprises—in addition to the catalytically active components—support material.

Catalyst precursors which comprise support material may comprise one or more catalytically active components, preferably CoO, NiO, Mn₃O₄, CuO and/or oxygen compounds of Rh, Ru and/or Ir.

The active composition of catalyst precursors which comprise support material more preferably comprises MO and/or CoO.

The support materials used are preferably carbon such as graphite, carbon black and/or activated carbon, aluminum oxide (gamma, delta, theta, alpha, kappa, chi or mixtures thereof), silicon dioxide, zirconium dioxide, zeolites, aluminosilicates, etc., and mixtures of these support materials.

The proportion of support material in the active composition may vary over a wide range according to the preparation method selected.

In the case of catalyst precursors which are prepared by impregnation, the proportion of support material in the active composition is generally more than 50% by weight, preferably more than 75% by weight and more preferably more than 85% by weight.

In the case of catalyst precursors which are prepared by precipitation reactions such as coprecipitation or precipitative application, the proportion of support material in the active composition is generally in the range from 10 to 90% by weight, preferably in the range from 15 to 80% by weight and more preferably in the range from 20 to 70% by weight.

Such catalyst precursors which are obtained by precipitation reactions are, for example,

catalysts disclosed in EP-A-696572, whose catalytically active composition, before reduction with hydrogen, comprises from 20 to 85% by weight of ZrO₂, from 1 to 30% by weight of oxygen compounds of copper, calculated as CuO, from 30 to 70% by weight of oxygen compounds of nickel, calculated as NiO, from 0.1 to 5% by weight of oxygen compounds of molybdenum, calculated as MoO₃, and from 0 to 10% by weight of oxygen compounds of aluminum and/or of manganese, calculated as Al₂O₃ and MnO₂ respectively, for example the catalyst disclosed in loc. cit, page 8, with the composition of 31.5% by weight of ZrO₂, 50% by weight of NiO, 17% by weight of CuO and 1.5% by weight of MoO₃,
catalysts disclosed in EP-A-963 975, whose catalytically active composition, before reduction with hydrogen, comprises from 22 to 40% by weight of ZrO₂, from 1 to 30% by weight of oxygen compounds of copper, calculated as CuO, from 15 to 50% by weight of oxygen compounds of nickel, calculated as NiO, where the molar Ni:Cu ratio is greater than 1, from 15 to 50% by weight of oxygen compounds of cobalt, calculated as CoO, from 0 to 10% by weight of oxygen compounds of aluminum and/or of manganese, calculated as Al₂O₃ and MnO₂ respectively, and does not comprise any oxygen compounds of molybdenum, for example the catalyst A disclosed in loc. cit., page 17, with the composition of 33% by weight of Zr, calculated as ZrO₂, 28% by weight of Ni, calculated as NiO, 11% by weight of Cu, calculated as CuO, and 28% by weight of Co, calculated as CoO,
copper catalysts disclosed in DE-A-2445303, for example the precipitated copper catalyst disclosed in example 1 there, which is prepared by treating a solution of copper nitrate and aluminum nitrate with sodium bicarbonate and subsequent washing, drying and heat treatment of the precipitate, and has a composition of approx. 53% by weight of CuO and approx. 47% by weight of Al₂O₃, or
catalysts disclosed in WO 96/36589, especially those which comprise Ir, Ru and/or Rh and, as a support material, activated carbon.

The catalyst precursors may be prepared by known processes, for example by precipitation, precipitative application, impregnation.

In a preferred embodiment, catalyst precursors which are prepared by impregnation of support materials (impregnated catalyst precursors) are used in the process according to the invention.

The support materials which are used in the impregnation may, for example, be used in the form of powders or shaped bodies such as extrudates, tablets, spheres or rings. Support material suitable for fluidized bed reactors is preferably obtained by spray-drying.

Useful support materials include, for example, carbon such as graphite, carbon black and/or activated carbon, aluminum oxide (gamma, delta, theta, alpha, kappa, chi or mixtures thereof), silicon dioxide, zirconium dioxide, zeolites, aluminosilicates or mixtures thereof.

The abovementioned support materials can be impregnated by the customary processes (A. B. Stiles, Catalyst Manufacture—Laboratory and Commercial Preparations, Marcel Dekker, New York, 1983), for example by application of a metal salt solution in one or more impregnation stages. Useful metal salts generally include water-soluble metal salts such as the nitrates, acetates or chlorides of the abovementioned elements. Thereafter, the impregnated support material is generally dried and, if appropriate, calcined.

The impregnation can also be effected by the so-called “incipient wetness method”, in which the support material, according to its water absorption capacity, is moistened up to a maximum of saturation with the impregnation solution. The impregnation can, though, also be effected in supernatant solution.

In multistage impregnation processes, it is appropriate to dry between individual impregnation steps and, if appropriate, to calcine. Multistage impregnation should advantageously be employed when the support material is to be coated with metal salts in a relatively large amount.

To apply a plurality of metal components to the support material, the impregnation can be effected simultaneously with all metal salts or successively in any sequence of the individual metal salts.

In a further preferred embodiment, catalyst precursors are prepared by means of a combined precipitation (coprecipitation) of all of their components. To this and, in general, a soluble metal salt of the corresponding metal oxides and if appropriate a soluble compound of a support material are admixed with a precipitant in a liquid under hot conditions and with stirring until the precipitation is complete.

The liquid used is generally water.

Useful soluble metal salts of the corresponding metal oxides typically include the corresponding nitrates, sulfates, acetates or chlorides of the metals of groups 8 and/or 9 and/or 10 and/or 11 of the Periodic Table of the Elements (Periodic Table in the IUPAC version of 06.22.2007). Examples of such metals are Cu, Co, Ni and/or Fe, and also noble metals such as Rh, Ir, Ru, Pt, Pd and Re.

The water-soluble compounds of a support material used are generally water-soluble compounds of Al, Zr, Si, etc., for example the water-soluble nitrates, sulfates, acetates or chlorides of these elements.

Catalyst precursors may also be prepared by precipitative application.

Precipitative application is understood to mean a preparation method in which a sparingly soluble or insoluble support material is suspended in a liquid and then soluble metal salts of the corresponding metal oxides are added, which are then applied to the suspended support by adding a precipitant (for example described in EP-A2-1 106 600, page 4, and A. B. Stiles, Catalyst Manufacture, Marcel Dekker, Inc., 1983, page 15).

Useful sparingly soluble or insoluble support materials include, for example, carbon compounds such as graphite, carbon black and/or activated carbon, aluminum oxide (gamma, delta, theta, alpha, kappa, chi or mixtures thereof), silicon dioxide, zirconium dioxide, zeolites, aluminosilicates or mixtures thereof.

The support material is generally present in the form of powder or spall.

The liquid used, in which support material is suspended, is typically water.

Useful soluble metal salts of the corresponding metal oxides generally include the corresponding nitrates, sulfates, acetates or chlorides of the metals of groups 8 and/or 9 and/or 10 and/or 11 of the Periodic Table of the Elements (Periodic Table in the IUPAC version of 06.22.2007). Examples of such metals are Cu, Co, Ni and/or Fe, and also noble metals such as Rh, Ir, Ru, Pt, Pd and Re.

In the precipitation reactions, the type of soluble metal salts used is generally not critical. Since the principal factor in this procedure is the water solubility of the salts, one criterion is their good water solubility which is required for the preparation of these comparatively highly concentrated salt solutions. It is considered to be self-evident that, in the selection of the salts of the individual components, of course only salts with those anions which do not lead to disruption, whether by causing undesired precipitation reactions or by complicating or preventing precipitation by complex formation, are selected.

Typically, in the precipitation reactions, the soluble compounds are precipitated as sparingly soluble or insoluble, basic salts by adding a precipitant.

The precipitants used are preferably alkalis, especially mineral bases, such as alkali metal bases. Examples of precipitants are sodium carbonate, sodium hydroxide, potassium carbonate or potassium hydroxide.

The precipitants used may also be ammonium salts, for example ammonium halides, ammonium carbonate, ammonium hydroxide or ammonium carboxylates.

The precipitation reactions can be performed, for example, at temperatures of from 20 to 100° C., particularly from 30 to 90° C., especially from 50 to 70° C.

The precipitates obtained in the precipitation reactions are generally chemically inhomogeneous and generally comprise mixtures of the oxides, oxide hydrates, hydroxides, carbonates and/or hydrogencarbonates of the metals used. It may be found to be favorable for the filterability of the precipitates when they are aged, i.e. when they are left for a certain time after the precipitation, if appropriate under hot conditions or while passing air through them.

The precipitates obtained by these precipitation processes are typically processed by washing, drying, calcining and conditioning them.

After washing, the precipitates are dried generally at from 80 to 200° C., preferably from 100 to 150° C., and then calcined.

The calcination is performed generally at temperatures between 300 and 800° C., preferably from 400 to 600° C., especially at from 450 to 550° C.

After the calcination, the catalyst precursors obtained by precipitation reactions are typically conditioned.

The conditioning can, for example, be effected by adjusting the precipitated catalyst to a particular particle size by grinding.

After the grinding, the catalyst precursor obtained by precipitation reactions can be mixed with shaping assistants such as graphite or stearic acid and processed further to give shaped bodies.

Common shaping processes are described, for example, in Ullmann [Ullmann's Encyclopedia Electronic Release 2000, chapter: “Catalysis and Catalysts”, pages 28-32] and by Ertl et al. [Ertl, Knözinger, Weitkamp, Handbook of Heterogeneous Catalysis, VCH Weinheim, 1997, pages 98 ff].

As described in the literature references mentioned, the shaping process can afford shaped bodies in any three-dimensional shape, for example round, angular, elongated or the like, for example in the form of extrudates, tablets, granule, spheres, cylinders or grains. Common shaping processes are, for example, extrusion, tableting, i.e. mechanical pressing or pelletizing, i.e. compaction by circular and/or rotating motions.

The conditioning or shaping is generally followed by a heat treatment. The temperatures in the heat treatment correspond typically to the temperatures in the calcination.

The catalyst precursors obtained by precipitation reactions comprise the catalytically active components in the form of a mixture of their oxygen compounds, i.e. especially as oxides, mixed oxides and/or hydroxides. The catalyst precursors thus prepared can be stored as such.

Before they are used as catalysts for the hydrogenating amination of glycerol, catalyst precursors which have been obtained by impregnation or precipitation as described above are generally prereduced by treatment with hydrogen after the calcination or conditioning.

For prereduction, the catalyst precursors are generally first exposed to a nitrogen-hydrogen atmosphere at from 150 to 200° C. over a period of from 12 to 20 hours, and then treated in a hydrogen atmosphere at from 200 to 400° C. for another up to approx. 24 hours. This prereduction reduces some of the oxygen-metal compounds present in the catalyst precursors to the corresponding metals, such that they are present together with the different types of oxygen compounds in the active form of the catalyst.

In a preferred embodiment, the prereduction of the catalyst precursor is undertaken in the same reactor in which the hydrogenating amination of the glycerol is subsequently performed.

The catalyst thus formed can, after the prereduction, be handled and stored under an inert gas such as nitrogen, or under an inert liquid, for example an alcohol, water or the product of the particular reaction for which the catalyst is used. The catalyst may, though, after the prereduction, also be passivated with an oxygen-comprising gas stream such as air or a mixture of air with nitrogen, i.e. be provided with a protective oxide layer.

The storage of the catalysts which have been obtained by prereduction of catalyst precursors under inert substances, or the passivation of the catalyst, enables uncomplicated and safe handling and storage of the catalyst. If appropriate, the catalyst must then be freed of the inert liquid before the start of the actual reaction, or the passivation layer must be removed, for example, by treatment with hydrogen or a hydrogen-comprising gas.

Before the start of the hydroamination, the catalyst can be freed of the inert liquid or passivation layer. This is done, for example, by treatment of the catalyst with hydrogen or a hydrogen-comprising gas. Preference is given to undertaking the hydroamination directly after the treatment of the catalyst in the same reactor in which the treatment of the catalyst with hydrogen or a hydrogen-comprising gas was also effected.

Catalyst precursors may, however, also be used in the process without prereduction, in which case they are then reduced by the hydrogen present in the reactor under the conditions of the hydrogenating amination, the catalyst generally being formed in situ.

The hydrogenating amination can be performed, for example, in a stirred autoclave, a bubble column, a circulation reactor, for instance a jet loop, or a fixed bed reactor. The process according to the invention can be performed batchwise or preferably continuously.

The hydrogenating amination of glycerol can be performed in the liquid phase or in the gas phase. Preference is given to performing the hydrogenating amination of glycerol in the liquid phase.

In the batchwise hydrogenating amination, a suspension of glycerol and catalyst is typically initially charged in the reactor. In order to ensure a high conversion and high selectivity, the suspension of glycerol and catalyst must generally be mixed well with hydrogen and the aminating agent, for example by means of a turbine stirrer in an autoclave. The suspended catalyst material can be introduced and removed again with the aid of customary techniques (sedimentation, centrifugation, cake filtration, crossflow filtration). The catalyst can be used once or more than once. The catalyst concentration is advantageously from 0.1 to 50% by weight, preferably from 0.5 to 40% by weight, more preferably from 1 to 30% by weight, especially from 5 to 20% by weight, based in each case on the total weight of the suspension consisting of glycerol and catalyst. The mean catalyst particle size is advantageously in the range from 0.001 to 1 mm, preferably in the range from 0.005 to 0.5 mm, especially from 0.01 to 0.25 mm.

If appropriate, the reactants can be diluted with a suitable inert solvent in which glycerol has a good solubility, such as tetrahydrofuran, dioxane, N-methylpyrrolidone.

In the continuous reductive amination, glycerol including hydrogen and aminating agent (ammonia or amine) is typically passed over the catalyst which is preferably disposed in a (preferably externally) heated fixed bed reactor.

In the case of performance of the process in the liquid phase, both trickle mode and upflow mode are possible.

The catalyst hourly space velocity is generally in the range from 0.05 to 5 kg, preferably from 0.1 to 2 kg, more preferably from 0.2 to 0.6 kg of glycerol per liter of catalyst (bed volume) and hour.

In the case of performance of the process in the liquid phase, the pressure is generally from 5 to 40 MPa (50-400 bar), preferably from 10 to 30 MPa, more preferably from 15 to 25 MPa.

The temperature is generally from 100 to 400° C., preferably from 150 to 300° C., more preferably from 180 to 250° C.

If appropriate, the reactants can be diluted with a suitable inert solvent in which glycerol has a good solubility, such as tetrahydrofuran, dioxane, N-methylpyrrolidone.

In the case of performance of the process in the gas phase, the gaseous reactants (glycerol plus aminating agent) are typically passed over the catalyst in the presence of hydrogen in a gas stream whose size has been selected so as to be sufficient for evaporation.

In the case of performance of the process in the gas phase, the pressure is generally from 0.01 to 40 MPa (0.1-400 bar), preferably from 0.1 to 10 MPa, more preferably from 0.1 to 5 MPa.

The temperature is generally from 100 to 400° C., preferably from 150 to 300° C., more preferably from 180 to 250° C.

It is also possible to employ a continuous suspension method, as described, for example, in EP-A2-1 318 128 (BASF AG) or in FR-A-2 603 276 (Inst. Français du Pétrole).

It is appropriate to heat the reactants even before they are supplied to the reaction vessel, preferably to the reaction temperature.

The aminating agent is preferably used in from 0.90 to 250 times the molar amount, more preferably from 1.0 to 100 times the molar amount, especially in from 1.0 to 10 times the molar amount, based in each case on glycerol.

Especially ammonia is generally with a from 1.5- to 250-fold, preferably from 2- to 100-fold, especially from 2- to 10-fold molar excess per mol of glycerol. Higher excesses both of ammonia and of primary or secondary amines are possible.

In addition, water can be used in the process according to the invention. Water can, for example, be supplied to the process together with glycerol in the form of an aqueous glycerol solution, but it can also be supplied to the reactor separately from the other starting materials.

Typically, the molar ratio of water to glycerol is less than 10:1, preferably less than 8:1. In a particular embodiment, no additional water is supplied to the process.

The hydrogen is supplied to the reaction generally in an amount of from 5 to 400 l, preferably in an amount of from 150 to 600 l per mol of glycerol, the liter data each having been converted to standard conditions (S.T.P.).

Both in the case of performance of the process in the liquid phase and in the case of performance of the process in the gas phase, the application of higher temperatures and higher total pressures is possible. The pressure in the reaction vessel, which arises from the sum of the partial pressures of the aminating agent, of glycerol and of the reaction products formed, and if appropriate of the solvent used, at the given temperatures, is appropriately increased to the desired reaction pressure by injecting hydrogen.

Both in the case of continuous performance of the process in the liquid phase and in the case of continuous performance of the process in the gas phase, the excess aminating agent can be circulated together with the hydrogen.

When the catalyst is arranged as a fixed bed, it may be advantageous for the selectivity of the reaction to mix the shaped catalyst bodies with inert random packings in the reactor, and in effect to “dilute” them. The proportion of the random packings in such catalyst formulations may be from 20 to 80, particularly from 30 to 60 and especially from 40 to 50 parts by volume.

The water of reaction formed in the course of the reaction (in each case one mol per mol of alcohol group converted) generally does not have a disruptive effect on the conversion, the reaction rate, the selectivity and the catalyst lifetime, and is therefore appropriately not removed from the reaction product until it is worked up, for example by distillation or extraction.

The process according to the invention can prepare amines from glycerol, hydrogen and an aminating agent selected from the group of ammonia and primary and secondary amine.

When the aminating agent used is ammonia, a reaction effluent comprising

one or more monoamines selected from the group consisting of methylamine, ethylamine, isopropylamine and n-propylamine, and/or one or more diamines selected from the group consisting of ethylenediamine, 1,2-propanediamine and 1,3-propanediamine, and/or one or more alkanolamines selected from the group consisting of monoethanolamine, 2-aminopropan-1-ol and 1-aminopropan-2-ol, preferably 2-aminopropan-1-ol, and/or one or more glycerol-like specialty amines selected from the group consisting of 1,2,3-triaminopropane, 1,3-diaminopropan-2-ol, 1,2-diaminopropan-3-ol, 1-aminopropanediol and 2-aminopropanediol, preferably 1,2,3-triaminopropane, 1,2-diaminopropan-3-ol and 2-aminopropanediol, and/or piperazine, and/or one or more piperazine derivatives selected from the group consisting of 2-methylpiperazine, 2,6-dimethylpiperazine, 2,5-dimethylpiperazine, 2,5-bis(aminomethyl)piperazine, 2,6-bis(aminomethyl)piperazine, 2-aminomethyl-5-methylpiperazine and 2-aminomethyl-6-methylpiperazine, preferably 2-aminomethyl-6-methylpiperazine, 2,5-bis(aminomethyl)piperazine and 2,6-bis(aminomethyl)piperazine is generally obtained.

Accordingly, the term “glycerol-like specialty amines” is understood to mean those amines which are characterized in that at least one hydroxyl group of glycerol has been substituted for a primary amino group, a secondary amino group or a tertiary amino group.

When ammonia is used as the aminating agent, one hydroxyl group of glycerol is substituted for a primary amino group.

When a primary amine is used as the aminating agent, for example methylamine, one hydroxyl group of glycerol is substituted for a secondary amino group, for example aminomethyl.

When a secondary amine is used as the aminating agent, for example dimethylamine, one hydroxyl group of glycerol is substituted for a tertiary amino group, for example aminodimethyl.

The composition of the reaction effluent can be influenced through the glycerol conversion, the reaction temperature and the composition of the catalyst.

For example, the composition of the catalyst used can influence the composition of the amines in the reaction effluent.

In a particular embodiment of the process according to the invention, the catalyst used is a catalyst which comprises Ni and/or Co, for example a Raney nickel or cobalt catalyst or a catalyst which has been obtained by reducing a catalyst precursor and whose active composition before the reduction with hydrogen comprises NiO and/or CoO as the catalytic active component. Such catalysts generally have a high activity and promote especially the formation of alkanolamines, diamines, glycerol-based specialty amines and/or piperazine derivatives.

In the case of catalysts which are prepared by reducing catalyst precursors, especially the presence of the catalytically active component NiO promotes the formation of glycerol-like specialty amines.

Preference is also given to an embodiment in which the catalyst used is a Cu catalyst. The use of Cu catalysts leads typically to a relatively high proportion of piperazine derivatives and/or diamines in the reaction effluent.

In a likewise preferred embodiment, catalysts which comprise one metal or a plurality of metals of the 5th period of groups 8 and/or 9 and/or 10 and/or 11 of the Periodic Table of the Elements, preferably Ru and/or Rh, are used in the process according to the invention. The use of such catalysts generally leads preferentially to formation of monoamines such as methylamine, ethylamine and/or isopropylamine when ammonia is used as the aminating agent.

In a further preferred embodiment, Raney sponge catalysts with Ni or Co as the active metal are used in the process according to the invention.

Raney sponge catalysts with Ni or Co as the active metal generally promote the formation of acyclic diamines and/or glycerol-like specialty amines. Since these catalysts have particularly high activity, a high yield of glycerol-like specialty amines or industrially important amines such as propanediamine and ethylenediamine is obtained even at low temperatures or with short reaction times when ammonia is used as the aminating agent.

A further preferred embodiment relates to the use of catalysts which comprise Ir in the process according to the invention. Catalysts which comprise Ir lead generally to a relatively high proportion of glycerol-like specialty amines.

It is also possible to influence the composition of the reaction effluent through the reaction temperature.

For example, with the same conversion at a lower reaction temperature, the formation of glycerol-based specialty amines and acyclic amines such as diamines and alkanolamines is generally promoted.

When the reaction is performed at a higher reaction temperature up to the same conversion, generally piperazine or piperazine derivatives are formed by cyclization reactions. In the case of a higher reaction temperature, deamination reactions generally increase at the same conversion; for example, piperazine is formed from aminomethylpiperazine or monoethylamine is formed from ethylenediamine.

The composition of the reaction effluent can also be influenced through the glycerol conversion.

For instance, it is observed that, in the case of high conversions, irrespective of the catalyst used, the number of functionalities generally decreases (defunctionalization), i.e., for example, di- or monoamines are formed from triamines, or methylpiperazines from aminomethylpiperazines.

In addition, in the case of high glycerol conversions, an increase in the cyclization and, associated with this, the formation of piperazine or piperazine derivatives is observed.

High glycerol conversions, for example glycerol conversions of 80% and more, preferably 90% and more, more preferably 99% and more, generally promote the formation of reaction effluents having a high proportion of cyclic amines, such as piperazine and/or piperazine derivatives.

Moderate glycerol conversions, for example glycerol conversions of from 30 to 80%, preferably from 40 to 70% and more preferably from 50 to 60%, generally promote the formation of glycerol-like specialty amines.

The glycerol conversion can be influenced by a series of process parameters such as pressure, temperature, the molar ratio of aminating agent, especially ammonia, relative to glycerol, and the reaction time or residence time.

The glycerol conversion (C_{glycerol}) can be determined as a matter of routine by gas chromatography analysis by the person skilled in the art and is typically reported as follows:

C_{glycerol}=(A %_{glycerol,start}−A %_{glycerol,end})/A %_{glycerol,start};

where A %_{glycerol, start} and A %_{glycerol, end} are the area percentages, determined by means of gas chromatography, below the glycerol signal which was measured at the start and end of the reaction or at the inlet and outlet of the reactor.

High glycerol conversions, for example from 80 to 100%, can, for example, be achieved by an increase in the temperature or an increase in the molar ratio of aminating agent, especially ammonia, relative to glycerol.

For example, high glycerol conversions can be achieved within a temperature range of generally from 200 to 400° C., preferably from 220 to 350° C.

Typically, the molar ratio of aminating agent, especially ammonia, relative to glycerol to achieve high glycerol conversions is in the range from 5:1 to 250:1, preferably from 10:1 to 150:1.

In a continuous process, it is possible to bring about a relatively high glycerol conversion by a reduction in the catalyst hourly space velocity.

High glycerol conversions are generally achieved at catalyst hourly space velocities in the range of from 0.05 to 0.6 kg of glycerol per liter of catalyst (bed volume) and hour, preferably from 0.05 to 0.2 per liter of catalyst (bed volume) and hour.

It is additionally possible to bring about a high glycerol conversion in a batchwise process by an increase in the residence time or by an increase in the catalyst concentration. Typically, high glycerol conversions are achieved in the case of residence times of from 16 to 72 hours, preferably from 20 to 48 hours and more preferably from 24 to 32 hours, where the residence time may also be shorter or longer depending on the catalyst concentration in order to achieve a high glycerol conversion.

Moderate glycerol conversions, for example from 30 to 80%, can, for example, be achieved by a reduction in the temperature or a reduction in the molar ratio of ammonia relative to glycerol.

Moderate glycerol conversions can be achieved, for example, within a temperature range of generally from 150 to 300° C., preferably from 150 to 220° C.

Typically, the molar ratio of aminating agent, especially ammonia, relative to glycerol in the case of achievement of high conversions is in the range from 1:1 to 100:1, preferably from 2.5:1 to 50:1.

It is possible to bring about a reduction in the glycerol conversion in a continuous process by an increase in the catalyst hourly space velocity. Moderate glycerol conversions are generally achieved at catalyst hourly space velocities in the range from 0.1 to 1.2 kg of glycerol per liter of catalyst (bed volume) and hour, preferably from 0.2 to 0.6 per liter of catalyst (bed volume) and hour.

In a batchwise process, it is possible to reduce the glycerol conversion by a shortening of the residence time or by a reduction in the catalyst concentration. Moderate glycerol conversions are generally achieved in the case of residence times of from 5 to 20 hours, preferably from 10 to 16 hours, where the residence time may also be shorter or longer depending on the catalyst concentration in order to achieve a moderate glycerol conversion.

In a particularly preferred embodiment, a catalyst comprising Ni and/or Co is used and a moderate glycerol conversion, for example a glycerol conversion of from 30 to 80%, preferably from 40 to 70% and more preferably from 50 to 60% is established.

A moderate glycerol conversion can typically be established as described above.

In the case of a moderate glycerol conversion and the use of nickel and/or cobalt catalysts, a high proportion of glycerol-based specialty amines is generally formed preferentially.

The reaction effluent obtained by this particular embodiment of the process comprises a proportion of glycerol-like specialty amines of generally more than 5% by weight, preferably more than 10% by weight, based on the total mass of the amines formed. In particular, the reaction effluent prepared by this embodiment of the process comprises 1,2,3-triaminopropane when ammonia is used as the aminating agent.

In a very particularly preferred embodiment, an Ir catalyst is used and a moderate glycerol conversion is established.

The establishment of a moderate glycerol conversion, for example from 30 to 80%, can generally be achieved in the manner detailed above.

The reaction effluent obtained by this particular embodiment of the process comprises a proportion of glycerol-like specialty amines of generally more than 5% by weight, preferably more than 10% by weight, based on the total mass of the amines formed.

In a further preferred embodiment, when a catalyst comprising Ni and/or Co is used, a high glycerol conversion, for example more than 80%, preferably more than 90%, more preferably more than 99%, is established.

High glycerol conversions can be established, for example, as described above.

The reaction effluent obtained by this particular embodiment of the process comprises a proportion of piperazine and/or piperazine derivatives of generally more than 10% by weight, preferably from 20% by weight to 80% by weight and more preferably from 30 to 70% by weight, based on the total mass of the amines formed.

In a further preferred embodiment, a catalyst which comprises one metal or a plurality of metals of the 5th period of groups 8 and/or 9 and/or 10 and/or 11 is used and a high glycerol conversion is established.

The establishment of a high glycerol conversion, for example more than 80%, can generally be achieved in the manner detailed above.

The reaction effluent obtained by this particular embodiment of the process comprises a proportion of monoamines of generally more than 30% by weight based on the total mass of the amines formed.

In a very particularly preferred embodiment, an Ni and/or Co catalyst is used within a temperature range of from 150 to 220° C. In this preferred embodiment, a high glycerol conversion, for example more than 80%, preferably more than 90%, more preferably more than 99%, is established, for example by reducing the catalyst hourly space velocity or increasing the residence time. The very particular embodiment differs from the embodiment specified above using Ni and/or Co catalysts at a high glycerol conversion in that a high glycerol conversion is established at temperatures in the range of 150 and 220° C. instead of from 220 to 350° C.

In the continuous variant of this very particular embodiment, the catalyst hourly space velocity is generally in the range from 0.05 to 0.6 kg of glycerol per liter of catalyst (bed volume) and hour, preferably from 0.05 to 0.2 per liter of catalyst (bed volume) and hour.

In the batchwise variant of this very particular embodiment, a high glycerol conversion can generally be achieved by prolonging of the residence time or by an increase in the catalyst concentration. Typically, the residence time in this particularly preferred embodiment is at a residence time of more than 20 hours, preferably more than 24 hours and more preferably more than 30 hours, where the residence time may also be shorter or longer depending on the catalyst concentration.

Typically, the molar ratio of aminating agent, especially ammonia, relative to glycerol to achieve high glycerol conversions in this very particular embodiment is in the range from 5:1 to 250:1, preferably from 10:1 to 150:1.

The reaction effluent obtained by this very particular embodiment of the process comprises a proportion of diaminopropane, diaminopropanol and triaminopropane of more than 10% by weight, preferably from 20% by weight to 80% by weight and more preferably from 30 to 70% by weight, based on the total mass of the amines.

The reaction effluent generally comprises the amines prepared in accordance with the invention, and also water, aminating agent, hydrogen and any unconverted glycerol.

Once the reaction effluent has appropriately been decompressed, the excess aminating agent and the hydrogen are removed therefrom. The excess aminating agent and the hydrogen are advantageously recycled back into the reaction zone.

Once the aminating agent and the hydrogen have been removed, the reaction effluent thus obtained is generally worked up.

In general, the reaction effluent is dewatered, since water and amines can form azeotropes which can complicate the distillative separation of the individual amines of the reaction effluent.

The aqueous reaction effluent is typically dewatered by contacting the aqueous reaction effluent with sodium hydroxide solution.

The concentration of the sodium hydroxide solution is typically from 20 to 80%, preferably from 30 to 70% and more preferably from 40 to 60%.

The volume ratio of added sodium hydroxide solution and the reaction effluent is typically between 0.5:1 to 2:1, preferably 1:1.

The reaction effluent can be contacted with sodium hydroxide solution by supplying the sodium hydroxide solution to the reaction reactor in which the hydrogenating amination of glycerol has been performed beforehand. In the case of a continuous reaction, the sodium hydroxide solution can be metered in as a continuous stream at the reactor outlet. However, it can also be contacted with the vaporous reaction effluent in the sense of an extractive distillation in a distillation column in countercurrent. Processes for extractive distillation are described, for example, in GB-A-1,1,02,370 or EP-A-1312600.

In a preferred variant, the reaction effluent is dewatered, for example, when a moderate glycerol conversion is established, since glycerol, in general, can typically be removed completely or virtually completely from the amines formed together with the aqueous phase.

The reaction effluent can be separated by distillation or rectification, liquid extraction or crystallization, and the separation can be effected in one or more stages, the number of stages generally being dependent on the number of components present in the reaction effluent.

The reaction effluent can be separated into fractions which comprise a mixture of different amine components, or into fractions which comprise only one amine component.

For example, a separation can first be effected into fractions which comprise more than one amine component. These fractions can subsequently, for example by a fine distillation, be separated into the individual compounds or components.

Unconverted glycerol can be recycled into the process.

The fractions obtained in the workup of the reaction effluent, comprising one or more amines, may, for example, be used as additives in concrete and/or cement production. Such fractions comprise, for example:

from 0 to 5% by weight of diamines such as 1,2-diaminopropane;
from 5 to 20% by weight of piperazine derivatives such as 2-methylpiperazine, 2,5-bis(aminomethyl)piperazine, 3,5-bis(aminomethyl)piperazine, 2-aminomethyl-6-methyl-piperazine, 3-aminomethyl-5-methylpiperazine and/or 3-aminomethyl-6-methyl-piperazine;
from 10 to 30% by weight of glycerol-like specialty amines such as 1,2,3-triaminopropane, 1,2-diaminopropan-3-ol and/or 1,3-diaminopropan-2-ol and from 20 to 45% by weight of glycerol.

In addition, such a fraction may comprise from 15 to 30% by weight of water and further components such as monoamines, diamines, piperazine, piperazine derivatives and/or alkanolamines.

The amines obtained in accordance with the invention, such as monoamines selected from the group consisting of methylamine, ethylamine, isopropylamine and n-propylamine, or

diamines such as ethylenediamine, 1,2-propanediamine and 1,3-propanediamine, or alkanolamines such as monoethanolamine, 2-aminopropan-1-ol and 1-aminopropan-2-ol, or
glycerol-like specialty such as 1,2,3-triaminopropane, 1,3-diaminopropan-2-ol, 1,2-diaminopropan-3-ol, 1-aminopropanediol and 2-aminopropanediol, or piperazine, or piperazine derivatives such as 2-methylpiperazine, 2,6-dimethylpiperazine, 2,5-dimethylpiperazine, 2,5-bis(aminomethyl)piperazine, 2,6-bis(aminomethyl)piperazine, 2-aminomethyl-5-methylpiperazine and 2-aminomethyl-6-methylpiperazine,
may be used as a synthesis unit for the production of surfactants, medicaments and crop protection compositions, stabilizers including light stabilizers, polymers, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators and/or emulsifiers.

The present invention further relates to the compound 1,2,3-triaminopropane of the general formula (I)

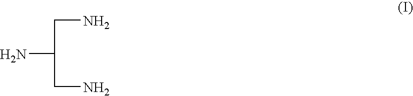

the compound 2-aminomethyl-6-methylpiperazine of the general formula (II)

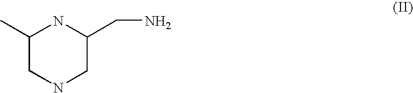

and the compound 2,5-bis(aminomethyl)piperazine or 2,6-bis(aminomethyl)piperazine of the general formula (III) or (IV).

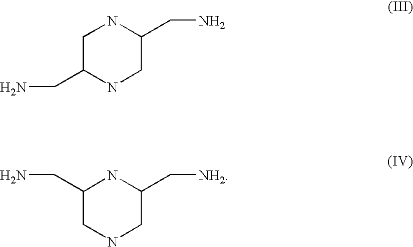

1,2,3-Triaminopropane is preferably obtained by hydrogenating amination of glycerol with ammonia using an Ir catalyst or an Ni and/or cobalt catalyst at moderate glycerol conversions. Moderate glycerol conversions can be established in the manner described above.

In a further preferred embodiment, 1,2,3-triaminopropane is obtained by using an Ni and/or Co catalyst at high glycerol conversions within a temperature range of from 150 to 220° C. as described above.

2-Aminomethyl-6-methylpiperazine, 2,5-bis(aminomethyl)piperazine or 2,6-bis(amino-methyl)piperazine are preferably obtained by hydrogenating amination of glycerol with ammonia using an Ni and/or cobalt catalyst or a Cu catalyst at high glycerol conversions. High glycerol conversions can be established in the manner described above.

The advantages of the present invention consist in the fact that glycerol is utilized effectively as a source for the preparation of amines. A process is provided which allows both important industrial amines and glycerol-based specialty amines, and also piperazine derivatives, to be obtained in order to utilize the glycerol raw material optimally.

The conversion of glycerol includes only a few reaction steps.

By virtue of easy-to-perform adjustments to the process conditions, for example pressure and temperature, and by virtue of the selection of the catalyst, it is possible to regulate the composition of the reaction effluent within certain limits in order to be able to react flexibly to variations in demand and sales.

This process can afford important industrial amines, for example monoamines such as methylamine, ethylamine, isopropylamine or n-propylamine, diamines such as ethylenediamine, 1,2-propanediamine or 1,3-propanediamine, alkanolamines such as monoethanolamine, 2-aminopropan-1-ol or 1-aminopropan-2-ol, or piperazine, which have to date been prepared from petrochemical starting materials. However, novel glycerol-based specialty amines are also obtained. Such amines are characterized in that at least one OH group of the glycerol has been substituted for a primary amino group, a secondary amino group or a tertiary amino group, for example 1, 2,3-triaminopropane, 1,3-diaminopropan-2-ol, 1,2-diaminopropan-3-ol, 1-aminopropanediol or 2-aminopropanediol. These compounds display a high number of functionalities and may therefore constitute important intermediates in the synthesis of organic compounds, such as crop protection compositions, pharmaceuticals, stabilizers, etc.

In addition, derivatives of piperazine (piperazine derivatives) such as 2-methylpiperazine, 2,6-dimethylpiperazine, 2,5-dimethylpiperazine, 2,5-bis(aminomethyl)piperazine, 2,6-bis(aminomethyl)piperazine, 2-aminomethyl-5-methyl-piperazine or 2-aminomethyl-6-methylpiperazine are obtained, which may likewise constitute important synthesis units.

The process according to the invention is illustrated by the examples which follow.

### EXAMPLES 1 to 10

### General Procedure

A high-pressure autoclave was charged with 5 g of pulverulent catalyst, 15 g of glycerol (glycerol puriss. 99.0-101.0% (pharmaceutical quality (Ph. Eur.)) from Riedel-de-Haën) and water. The reaction was inertized and ammonia was added. The inert gas was exchanged for hydrogen and then hydrogen was injected to a pressure of 20 bar. The reactor was heated with stirring to the end temperature within 2 hours and, on attainment of this temperature, the pressure was increased to 200 bar by injecting hydrogen. The pressure was kept constant at this value over the entire duration of the reaction. After a reaction time of 48 hours, the reactor was cooled to room temperature and decompressed slowly at room temperature. The degassed reactor contents were analyzed. The contents of the compounds were determined by means of gas chromatography (conditions: RTX 5 Amine 30 m capillary column, film thickness 1.5 micrometers, diameter 0.32 mm, method: 5 min at 60° C., then heat to 280° C. at 7° C./min and continue heating at 280° C. for 20 min) as area percentages (A %). In this context, the area percentages of the signals are based on the total area below the signals measured with the exception of the water signal.

The glycerol conversions reported are based on the area percentages determined before the start and at the end of the reaction.

### Example 1

The preparation was effected as described in the general procedure. The catalyst used was a Raney nickel catalyst. 22.5 g of water and 90 g of ammonia were used. The end temperature was 200° C.

Samples were taken after various reaction times.

After 32 hours, gas chromatography analysis gave the following composition:

ethylenediamine: 8%; 1,2-propylenediamine: 22%; piperazine: 2%; 2-methylpiperazine: 13%; 2,6-dimethylpiperazine: 11%; 1,2-diaminopropan-3-ol: 21%; 1,2,3-triaminopropane: 2%. The glycerol conversion was 91%.

Gas chromatography analysis after 48 hours gave the following composition:

ethylenediamine: 1%; 1,2-propylenediamine: 17%; piperazine: 3%; 2-methylpiperazine: 25%; 2,6-dimethylpiperazine: 31%; 1,2-diaminopropan-3-oI: 4%; 1,2,3-triaminopropane: 0%

The glycerol conversion was approx. 100%.

### Example 2

The preparation was effected as described in the general procedure. The catalyst used was Raney cobalt (Raney® 2724 from Grace Davison). 11.25 g of water and 90 g of ammonia were used. The end temperature was 200° C. Samples were taken after various reaction times.

After 16 hours, gas chromatography analysis gave the following composition:

ethylenediamine: 5%; 1,2-propylenediamine: 22%; piperazine: 3%; 2-methylpiperazine: 17%; 2,6-dimethylpiperazine: 11%; 1,2-diaminopropan-3-ol: 8%; 1,2,3-triaminopropane: 0%

After 48 hours, gas chromatography analysis gave the following composition:

ethylenediamine: 0%; 1,2-propylenediamine: 2%; piperazine: 1%; 2-methylpiperazine: 12%; 2,6-dimethylpiperazine: 37%; 1,2-diaminopropan-3-ol: 6%; 1,2,3-triaminopropane: 0%

The glycerol conversion was approx. 100%.

### Example 3

The preparation was effected as described in the general procedure. The catalyst used was a catalyst which was obtained by prereduction from a catalyst precursor whose catalytically active composition before the reduction with hydrogen 13% by weight of Cu, calculated as CuO, 28% by weight of Ni, calculated as NiO, 28% by weight of Co, calculated as CoO and 31% by weight of Zr, calculated as ZrO₂.

The catalyst precursor was prereduced at a temperature of 280° C. under a pure hydrogen atmosphere for 20 hours.

22.5 g of water and 90 g of ammonia were used. The end temperature was 200° C.

The gas chromatography analysis gave the following composition:

ethylenediamine: 1%; 1,2-propylenediamine: 17%; piperazine: 6%; 2-methylpiperazine: 37%; 2,6-dimethylpiperazine: 27%; 1,2-diaminopropan-3-ol: 7%; 1,2,3-triaminopropane: 0%

The glycerol conversion was approx. 100%.

### Example 4

The preparation was effected analogously to example 3, except that only 11.25 g instead of 22.5 g of water were used in the reaction.

The gas chromatography analysis gave the following composition:

ethylenediamine: 8%; 1,2-propylenediamine: 36%; piperazine: 0%; 2-methylpiperazine: 3%; 2,6-dimethylpiperazine: 15%; 1,2-diaminopropan-3-ol: 3%; 1,2,3-triaminopropane: 0%

The glycerol conversion was approx. 100%.

### Example 5

The preparation was effected analogously to example 3, except that the reaction time was only 38 hours instead of 48 hours.

The gas chromatography analysis gave the following composition:

ethylenediamine: 2%; 1,2-propylenediamine: 16%; piperazine: 0%; 2-methylpiperazine: 2%; 2,6-dimethylpiperazine: 3%; 1,2-diaminopropan-3-ol: 10%; 1,2,3-triaminopropane: 13%

The glycerol conversion was approx. 77%.

### Example 6

The preparation was effected analogously to example 3, except that the reaction temperature was 240° C. instead of 200° C.

The gas chromatography analysis gave the following composition:

ethylenediamine: 3%; 1,2-propylenediamine: 27%; piperazine: 3%; 2-methylpiperazine: 19%; 2,6-dimethylpiperazine: 20%; 1,2-diaminopropan-3-ol: 5%; 1,2,3-triaminopropane: 0%

The glycerol conversion was approx. 100%.

### Example 7

The preparation was effected analogously to example 3, except that the reaction temperature was 190° C. instead of 200° C. and the amount of ammonia used was 78 g instead of 90 g.

The gas chromatography analysis gave the following composition:

ethylenediamine: 0%; 1,2-propylenediamine: 17%; piperazine: 1%; 2-methylpiperazine: 16%; 2,6-dimethylpiperazine: 33%; 1,2-diaminopropan-3-ol: 5%; 1,2,3-triaminopropane: 0%

The glycerol conversion was approx. 100%.

### Example 8

The preparation was effected as described in the general procedure. The catalyst used was a catalyst which had been obtained by prereduction from a catalyst precursor whose catalytically active composition before the reduction with hydrogen comprised 50% by weight of Ni, calculated as NiO, 18% by weight of Cu, calculated as CuO, 2% by weight of Mo, calculated as MoO₃, and 30% by weight of Zr, calculated as ZrO₂.

The catalyst precursor was prereduced at a temperature of 280° C. under a pure hydrogen atmosphere for 12 hours.

No water and 78 g of ammonia were used. The end temperature was 190° C.

The gas chromatography analysis gave the following composition:

ethylenediamine: 5%; 1,2-propylenediamine: 29%; piperazine: 1%; 2-methylpiperazine: 9%; 2,6-dimethylpiperazine: 7%; 1,2-diaminopropan-3-ol: 7%; 1,2,3-triaminopropane: 0%

The glycerol conversion was approx. 89%.

### Example 9

The preparation was effected as described in the general procedure. The catalyst used was a catalyst which had been obtained by prereduction from a catalyst precursor whose catalytically active composition before the reduction with hydrogen comprised 85% by weight of Co, calculated as CoO, and 5% by weight of Mn, calculated as Mn₃O₄.

The catalyst precursor was prereduced at a temperature of 280° C. under a pure hydrogen atmosphere for 12 hours.

11.25 g of water and 90 g of ammonia were used. The end temperature was 200° C.

The gas chromatography analysis gave the following composition: ethylenediamine: 3%; 1,2-propylenediamine: 12%; piperazine: 0%; 2-methylpiperazine: 1%; 2,6-dimethylpiperazine: 0%; 1,2-diaminopropan-3-ol: 1%; 1,2,3-triaminopropane: 0%; 2-aminopropanol: 10%

The glycerol conversion was approx. 41%.

### Example 10

The preparation was effected as described in the general procedure. The catalyst used was a catalyst which had been obtained by prereduction from a catalyst precursor whose catalytically active composition before the reduction with hydrogen comprised 39% by weight of Cu, calculated as CuO, and 30% by weight of Cr, calculated as Cr₂O₃. The catalyst precursor was prereduced at a temperature of 280° C. under a pure hydrogen atmosphere for 20 hours.

No water and 78 g of ammonia were used. The end temperature was 200° C.

The gas chromatography analysis gave the following composition:

ethylenediamine: 5%; 1,2-propylenediamine: 30%; piperazine: 2%; 2-methylpiperazine: 10%; 2,6-dimethylpiperazine: 15%; 1,2-diaminopropan-3-ol: 4%; 1,2,3-triaminopropane: 0%

The glycerol conversion was approx. 83%.

### ExampleS 11 TO 16

### General Procedure

A high-pressure autoclave was charged with 0.5 g of pulverulent catalyst, 4.8 g of glycerol (glycerol puriss. 99.0-101.0% (pharmaceutical quality (Ph. Eur.)) from Riedel-de-Haën) and 6.5 g of water. The reaction was inertized and ammonia was added. The inert gas was exchanged for hydrogen and then hydrogen was injected to a pressure of 50 bar. The reactor was heated with stirring to 250° C. within 2 hours and, on attainment of this temperature, the pressure was increased to 300 bar by injecting hydrogen. The pressure was kept constant at this value over the entire duration of the reaction. After a reaction time of 24 hours, the reactor was cooled to room temperature and decompressed slowly at room temperature. The degassed reactor contents were analyzed. The contents of the compounds were determined by means of gas chromatography (conditions: RTX 5 Amine 30 m capillary column, film thickness 1.5 micrometers, diameter 0.32 mm, method: 5 min at 60° C., then heat to 280° C. at 7° C./min and continue heating at 280° C. for 20 min) as area percentages (A %). In this context, the area percentages of the signals are based on the total area below the signals measured with the exception of the water signal.

The glycerol conversions reported are based on the area percentages determined before the start and at the end of the reaction.

### Example 11

The preparation was effected as described in the general procedure. The catalyst used was a catalyst which had been obtained by prereduction from a catalyst precursor whose catalytically active composition before the reduction with hydrogen comprised 13% by weight of Cu, calculated as CuO, 28% by weight of NI, calculated as NiO, 28% by weight of Co, calculated as CoO and 31% by weight of Zr, calculated as ZrO₂. The catalyst precursor was prereduced at a temperature of 280° C. under a pure hydrogen atmosphere for 20 hours.

The gas chromatography analysis gave the following composition:

methylamine: 4%; ethylamine: 9%; isopropylamine: 5%; n-propylamine: 6%; piperazine: 3%; 2-methylpiperazine: 9%; 1-aminopropan-2-ol and 2-amino-1-ol: 3%; 1,2-propanediamine: 8%; 1,2-diaminopropan-3-ol: 5%

The glycerol conversion was approx. 100%.

### Example 12

The preparation was effected as described in the general procedure. The catalyst used was a catalyst which had been obtained by prereduction from a catalyst precursor whose catalytically active composition consisted of LiCoO₂. The catalyst precursor was prereduced at a temperature of 300° C. under a pure hydrogen atmosphere for 20 hours.

The gas chromatography analysis gave the following composition:

methylamine: 0%; ethylamine: 1%; isopropylamine: 1%; n-propylamine: 1%; piperazine: 2%; 2-methylpiperazine: 17%; 1-aminopropan-2-ol and 2-amino-1-ol: 4%; 1,2-propanediamine: 6%; 1,2-diaminopropan-3-ol: 1%

The glycerol conversion was approx. 90%.

### Example 13

The preparation was effected as described in the general procedure. The catalyst used was a catalyst which had been obtained by prereduction from a catalyst precursor whose catalytically active composition consisted of RuO(OH)ₓ.

The gas chromatography analysis gave the following composition:

methylamine: 10%; ethylamine: 16%; isopropylamine: 12%; n-propylamine: 16%; piperazine: 0%; 2-methylpiperazine: 0%; 1-aminopropan-2-ol and 2-amino-1-ol: 0%; 1,2-propanediamine: 0%; 1,2-diaminopropan-3-ol: 0%

The glycerol conversion was approx. 100%.

### Example 14

The preparation was effected as described in the general procedure. The catalyst used was a catalyst which comprised 5% by weight of iridium on activated carbon.

The gas chromatography analysis gave the following composition:

methylamine: 0%; ethylamine: 0%; isopropylamine: 0%; n-propylamine: 0%; piperazine: 2%; 2-methylpiperazine: 6%; 1-aminopropan-2-ol and 2-amino-1-ol: 15%; 1,2-propanediamine: 2%; 1,2-diaminopropan-3-ol: 10% The glycerol conversion was approx. 100%.

### Example 15

The preparation was effected as described in the general procedure. The catalyst used was a catalyst which comprised 5% by weight of rhodium on activated carbon.

The gas chromatography analysis gave the following composition:

methylamine: 4%; ethylamine: 14%; isopropylamine: 20%; n-propylamine: 27%; piperazine: 0%; 2-methylpiperazine: 0%; 1-aminopropan-2-ol and 2-amino-1-ol: 0%; 1,2-propanediamine: 0%; 1,2-diaminopropan-3-ol: 0%

The glycerol conversion was approx. 100%.

### ExampleS 16 TO 29

### General Procedure

1 l of catalyst precursors were charged into a 1.2 tubular reactor between 100 ml in each case of V2A steel rings having a diameter of 6 mm.

The catalyst precursor used was a catalyst precursor whose active composition comprised 50% by weight of Ni, calculated as NiO, 18% by weight of Cu, calculated as CuO, 2% by weight of Mo, calculated as MoO₃, and 30% by weight or Zr, calculated as ZrO₂.

The catalyst precursor was heated at a temperature of 280° C. and a hydrogen feed of 200 l (STP)/h (I (STP): standard liters; h: hour) for 12 hours and then reduced at 280° C. at a hydrogen feed of 200 l (STP)/h for 24 hours.

Glycerol (99.8% pharmaceutical quality from Cognis), ammonia and hydrogen were passed continuously to the reactor in trickle mode in the amount specified in table 1. The reactor pressure was 200 bar. The temperature of the heat carrier oil at the reactor outlet is specified in table 1.

The structure of the piperazine derivatives separated by gas chromatography was determined by means of mass spectroscopy:

### 2-Aminomethylpiperazine

MS (EI+) from GC-MS

Method: 30 m RTX-5 amine (1.5 μm), 50/5-7-300/10. (K1, t_{R}=17.2 min.):

m/z (%)=115 (2) [M⁺], 98 (7) [M⁺-NH₃], 97 (2), 86 (5), 85 (77), 84 (4), 83 (4), 69 (4), 68 (6), 59 (2), 58 (6), 57 (12), 56 (100), 55 (12), 54 (5), 44 (16), 43 (7), 42 (12), 41 (7).

High-resolution MS (FI) from GC-MS

m/z (%)=231 (8) [2*M+H⁺], 116 (74)[M+H], 115 (100) [M⁺].

### 2-Aminomethyl-5-methylpiperazine

MS (EI+) from GC-MS

Method: 30 m RTX-5 amine (1.5 μm), 50/5-7-300/10. (K2, t_{R}=17.8 min.):

m/z (%)=129 (4) [M⁺], 112 (3) [M⁺-NH₃], 100 (6), 99 (100), 98 (3), 97 (2), 86 (4), 85 (5), 84 (1), 83 (5), 70 (55), 69 (33), 68 (18), 59 (5), 58 (13), 57 (7), 56 (73), 55 (3), 54 (14), 44 (45), 43 (13), 42 (23), 41 (16).

High-resolution MS (FI) from GC-MS

m/z (%)=259 (4) [2*M+H⁺], 130 (45) [M⁺+H], 129 (100) [M⁺].

### 2-Aminomethyl-5-methylpiperazine

MS (EI+) from GC-MS

Method: 30 m RTX-5 amine (1.5 μm), 50/5-7-300/10. (K3, t_{R}=18.0 min.):

m/z (%)=129 (4) [M⁺], 112 (4) [M⁺-NH₃], 100 (6), 99 (94), 98 (3), 97 (2), 84 (4), 83 (5), 82 (16), 73 (4), 71 (6), 70 (13), 69 (4), 68 (13), 59 (3), 58 (39), 57 (29), 56 (100), 55 (12), 54 (7), 44 (37), 43 (6), 42 (14), 41 (13).

High-resolution MS (FI) from GC-MS

m/z (%)=259 (5) [2*M+H⁺], 130 (44) [M⁺+H], 129 (100) [M⁺].

### 2-Aminomethyl-6-methylpiperazine

MS (EI+) from GC-MS

Method: 30 m RTX-5 amine (1.5 μm), 50/5-7-300/10. (K4, t_{R}=18.3 min.):

m/z (%)=129 (1) [M⁺], 112 (3) [M⁺-NH₃], 100 (7), 99 (98), 98 (4), 97 (2), 85 (3), 84 (1), 83 (5), 82 (6), 72 (7), 71 (8), 70 (39), 69 (19), 68 (13), 59 (4), 58 (15), 57 (11), 56 (100), 55 (4), 54 (11), 44 (48), 43 (11), 42 (21), 41 (14).

High-resolution MS (FI) from GC-MS

m/z (%)=259 (7) [2*M+H⁺], 130 (100) [M⁺+H], 129 (74) [M⁺].

### 2,5-Bis(aminomethyl)piperazine

MS (EI+) from GC-MS

Method: 30 m RTX-5 amine (1.5 μm), 50/5-7-300/10. (K7, t_{R}=23.1 min.):

m/z (%)=144 (1) [M⁺], 127 (5) [M⁺-NH₃], 115 (7), 114 (100), 98 (5), 97 (58), 95 (3), 86 (2), 85 (9), 84 (4), 83 (11), 82 (6), 80 (4), 71 (9), 70 (6), 69 (15), 68 (43), 67 (4), 59 (15), 58 (10), 57 (10), 56 (55), 55 (8), 54 (7).

High-resolution MS (FI) from GC-MS

m/z (%)=289 (12) [2*M+H⁺], 145 (100) [M⁺+H], 144 (76) [M⁺].

5080 g of the reaction effluent from example 29 were dewatered with 50% sodium hydroxide solution. The volume ratio of sodium hydroxide solution to reactor effluent was 1:1.

A separation was effected into an aqueous and an organic phase. Glycerol was removed together with the aqueous phase. The organic phase (3351 g) was distilled. The column used was a 1 m column with a diameter of 50 mm which had been filled with 3 mm V2A mesh rings. The number of theoretical plates was 20. The pressure was lowered from 200 mbar to 1 mbar and the bottom temperature was increased from 86 to 194° C. The reaction mixture was divided into different fractions. The bottom residue was 755 g.

The sequence of products distilled over was: ethylenediamine, 1,2-propylenediamine, 1,3-propylenediamine, methylpiperazine, 2,6-dimethylpiperazine, 1,2,3-triaminopropane, 1,2-diaminopropan-3-ol, followed by various isomers of aminomethylmethylpiperazine. A fine distillation of some fractions (525 g) afforded 180 g of 1,2,3-triaminopropane in 92% purity. The molecular structure of 1,2,3-triaminopropane was confirmed by high-resolution mass spectroscopy and NMR NMR (CDCl₃, 500 MHz): δ=1.32 (s, 6H, NH), 2.44 (dd, 2H, CH₂), 2.55-2.59 (m, 1H, CH), 2.67 (dd, 2H, CH₂). —¹³C NMR (CDCl₃, DEPT, 126 MHz): δ=46.5 (T CH₂), 56.1 (D, CH)).

### ExampleS 30 TO 32

### General Procedure

4 l of catalyst precursors were charged into a 5 l tubular reactor between in each case 500 ml of V2A steel rings having a diameter of 6 mm.

The catalyst precursor used was a catalyst precursor whose active composition comprised 13% by weight of Cu, calculated as CuO, 28% by weight of Ni, calculated as NiO, 28% by weight of Co, calculated as CoO and 31% by weight of Zr, calculated as ZrO₂.

The catalyst precursor was heated to a temperature of 280° C. at a hydrogen feed of 200 l (STP)/h for 12 hours and then reduced at a hydrogen feed of 200 l (STP)/h at 280° C. for 24 hours.

Glycerol, ammonia and hydrogen were passed continuously to the reactor from the top in trickle mode in the amount specified in table 2.

The reactor pressure was 200 bar. The temperature of the heat carrier oil at the reactor outlet is specified in table 2.

The contents of the compounds determined by means of gas chromatography (conditions: RTX 5 amine 30 m capillary column, film thickness 1.5 micrometers, diameter 0.32 mm, method: 60° C. for 5 min, then heat to 280° C. at 7° C./min and continue heating at 280° C. for 20 min) as area percentages (A %). In this context, the area percentages of the signals are based on the total area below the signals measured with the exception of the water signal.

The glycerol conversions reported are based on the area percentages determined before the start and at the end of the reaction and are likewise reported in table 2.

A portion of the effluent from example 32 was boiled under reflux at a bottom temperature of 120° C. and standard pressure in a distillation apparatus with a column (1 m column with a diameter of 50 mm, which was filled with 3 mm V2A mesh rings. The number of theoretical plates was 20) for 10 hours. In the course of this, ammonia was depleted down to a residual content of 20 ppm. The water content of the mixture was 23% (Karl-Fischer titration). The amine numbers were determined titrimetrically by the customary derivatization method. The primary amine number was 228 mg KOH/g; the secondary amine number 317 mg KOH/g and the tertiary amine number 16 mg KOH/g. This corresponds to a total amine number of 561 mg KOH/g.

The mixture was used as an additive in cement production.

TABLE 1

| | Reaction | | | | | Glycerol | 1,2- | 2-Amino- |
|---|---|---|---|---|---|---|---|---|
| | time | T | Ammonia | Glycerol | Hydrogen | conversion | PDA | propanol |
| Ex. | [h] | [° C.] | [g/h] | [g/h] | [I (STP)/h] | [%] | [%] | [%] |
| 16 | 61 | 180 | 510 | 92 | 300.00 | 64.3 | 7.4 | 6 |
| 17 | 109 | 190 | 510 | 92 | 300.00 | 79.6 | 12.3 | 5.2 |
| 18 | 157 | 200 | 510 | 92 | 300.00 | 87.5 | 16 | 2.4 |
| 19 | 205 | 210 | 510 | 92 | 300.00 | 91.9 | 14.4 | 1.6 |
| 20 | 253 | 190 | 255 | 92 | 300.00 | 65 | 9 | 3.1 |
| 21 | 301 | 190 | 765 | 92 | 300.00 | 81.7 | 12.2 | 5.6 |
| 22 | 349 | 190 | 1020 | 184 | 300.00 | 57.9 | 7.6 | 5.5 |
| 23 | 397 | 190 | 1530 | 276 | 300.00 | 45 | 6.1 | 5.2 |
| 24 | 469 | 190 | 510 | 92 | 300.00 | 71.2 | 11 | 5.3 |
| 25 | 649 | 190 | 510 | 92 | 150.00 | 78.4 | 12.9 | 4.5 |
| 26 | 757 | 190 | 380 | 69 | 100.00 | 88.5 | 16.1 | 3.6 |
| 27 | 829 | 200 | 380 | 69 | 100.00 | 95 | 19.8 | 1.9 |
| 28 | 901 | 190 | 280 | 50 | 100.00 | 94.9 | 17.8 | 1.9 |
| 29 | 1076 | 192 | 280 | 50 | 75.00 | 98.7 | 20.1 | 1.4 |

| | 1,2,3- | 1,2- | 2-Me- | 2,6-Di- | 2,5-Di- | Amino- | Bis-Amino- | |
|---|---|---|---|---|---|---|---|---|
| | TAP | DAPOL | Pip | Me-Pip | Me-Pip | Me-Pip | Me-Pip | Glycerol |
| Ex. | [%] | [%] | [%] | [%] | [%] | [%] | [%] | [%] |
| 16 | 12.6 | 0 | 0.3 | 2.2 | 6.2 | 14.5 | 1.6 | 35.7 |
| 17 | 10.2 | 0.9 | 0.9 | 4.8 | 3.8 | 20.5 | 1.6 | 20.4 |
| 18 | 5 | 3 | 2.6 | 13.8 | 1.4 | 17.1 | 0.7 | 12.5 |
| 19 | 2 | 5.4 | 5.5 | 28 | 0 | 7.5 | 0.3 | 8.1 |
| 20 | 6.1 | 1.8 | 1.5 | 8.8 | 2.6 | 12.5 | 0.6 | 35 |
| 21 | 12.6 | 0.9 | 0.8 | 4 | 3.8 | 21.3 | 1.8 | 18.3 |
| 22 | 12.1 | 0 | 0.4 | 2 | 6 | 10.8 | 1 | 42.1 |
| 23 | 11.6 | 0 | 0.2 | 1.2 | 6 | 5.9 | 0.5 | 55 |
| 24 | 13.6 | 0 | 0.6 | 3.6 | 6.8 | 15 | 2 | 28.8 |
| 25 | 14.3 | 1 | 0.8 | 3.8 | 3.9 | 19.5 | 1.8 | 21.6 |
| 26 | 14.7 | 1.2 | 1.1 | 4.8 | 2.8 | 24 | 2.3 | 11.5 |
| 27 | 6 | 3.8 | 3.9 | 17.8 | 0.9 | 16.8 | 0.7 | 5 |
| 28 | 8.1 | 2.2 | 2.1 | 10.3 | 1.2 | 24.6 | 1.4 | 5.1 |
| 29 | 6.7 | 2.6 | 2.6 | 11.7 | 0.4 | 26.4 | 1.3 | 1.3 |

Legend:
1,2-PDA = 1,2-diaminopropane;
2-aminopropanol = 2-aminopropan-1-ol;
1,2,3 TAP = 1,2,3-triaminopropane;
1,2-DAPOL = 1,2-diaminopropan-3-ol;
2-Me-Pip = 2-methylpiperazine;
2,6-di-Me-Pip = 2,6-dimethylpiperazine;
2,5-di-Me-Pip = 2,5-dimethylpiperazine;
amino-Me-Pip = 2-aminomethyl-6-methylpiperazine and/or 2-aminomethyl-5-methylpiperazine;
bis-amino-Me-Pip = 2,5-bis(aminomethyl)piperazine and/or 2,6-bis(aminomethyl)piperazine;

TABLE 2

| | Reaction | | | | Hydrogen | Glycerol | | 2-Amino- |
|---|---|---|---|---|---|---|---|---|
| | time | T | Ammonia | Glycerol | [I | Conversion | 1,2-PDA | propanol |
| Ex. | [h] | [° C.] | [g/h] | [g/h] | (STP)/h] | [%] | [%] | [%] |
| 30 | 53 | 180 | 1500 | 400 | 500.00 | 99.9 | 13.3 | 0.6 |
| 31 | 89 | 175 | 1500 | 800 | 500.00 | 83.3 | 5.8 | 6.3 |
| 32 | 185 | 174 | 2230 | 1200 | 500.00 | 63.1 | 3.3 | 5.9 |
| | 205 | 210 | 510 | 92 | 300.00 | 91.9 | 14.4 | |

| | | | | 1,2- | | Bis- | |
|---|---|---|---|---|---|---|---|
| | 2-Me- | 2,6-Di- | 1,2,3- | DAP-3- | | Amino- | Amino- |
| | Pip | Me-Pip | TAP | OL | Glycerol | Me-Pip | Me-Pip |
| Ex. | [%] | [%] | [%] | [%] | [%] | [%] | [%] |
| 30 | 2.3 | 7 | 6.3 | 1.4 | 0.2 | 3.1 | 23.7 |
| 31 | 0.8 | 2.9 | 10.1 | 9.7 | 16.7 | 3.6 | 14.3 |
| 32 | 0.3 | 2.1 | 9.6 | 15.4 | 36.9 | 2.2 | 7.7 |
| | 1.6 | 28 | 2 | 5.4 | 0.3 | 7.5 | |

Legend:
1,2-PDA = 1,2-diaminopropane;
2-aminopropanol = 2-aminopropan-1-ol;
2-Me-Pip = 2-methylpiperazine;
2,6-di-Me-Pip = 2,6-dimethylpiperazine;
1,2,3 TAP = 1,2,3-triaminopropane;
1,2-DAPOL = 1,2-diaminopropan-3-ol;
Bis-amino-Me-Pip = 2,5-bis(aminomethyl)piperazine and/or 2,6-bis(aminomethyl)piperazine;
amino-Me-Pip = 2-aminomethyl-6-methylpiperazine and/or 2-aminomethyl-5-methylpiperazine

1.-19. (canceled)

21. A process for preparing amines which comprises reacting glycerol with hydrogen and an aminating agent from the group of ammonia and primary and secondary amines in the presence of a catalyst at a temperature of from 100° C. to 400° C. and a pressure of from 0.01 to 40 MPa (from 0.1 to 400 bar), wherein the process further requires (a), (b) or (c):
(a) the glycerol conversion is in the range from 30 to 80%,
(b) the catalyst comprises Ir or
(c) the reaction effluent is dewatered before the distillative workup.

22. The process as claimed in claim 20, wherein the glycerol conversion is in the range from 30 to 80%.

23. The process as claimed in claim 20, wherein the catalyst comprises Ir.

24. The process as claimed in claim 20, wherein the reaction effluent is dewatered before the distillative workup.

25. The process according to claim 20, wherein the glycerol is glycerol based on renewable raw materials.

26. The process according to claim 20, wherein the catalyst comprises one metal or a plurality of metals or one or more oxygen compounds of the metals of groups 8 and/or 9 and/or 10 and/or 11 of the Periodic Table of the Elements.

27. The process according to claim 20, wherein the catalyst comprises Ni or NiO and/or Co or CoO.

28. The process according to claim 20, wherein the catalyst comprises Cu or CuO.

29. The process according to claim 20, wherein the catalyst comprises one metal or a plurality of metals of the 5th period of groups 8 and/or 9 and/or 10 and/or 11 of the Periodic Table of the Elements.

30. The process according to claim 20, wherein the catalyst is a Raney sponge catalyst comprising Ni or Co or a mixture thereof.

31. The process according to claim 20, wherein the catalyst hourly space velocity is in the range from 0.1 to 1.21 g of glycerol per liter of catalyst (bed volume) and hour, and the temperature is in the range from 150 to 220° C.

32. The process according to claim 23, wherein the reaction effluent, after distillative removal of the aminating agent, has an aminating agent content of less than 1000 ppm.

33. The process according to claim 32, wherein the aminating agent used is ammonia.

34. An additive in cement or concrete production which comprises the reaction effluent obtainable according to claim 32.

35. The process according to claim 32, wherein the reaction effluent is obtained, which comprises
one or more monoamines selected from the group consisting of methylamine, ethylamine, isopropylamine and n-propylamine, and/or
one or more diamines selected from the group consisting of ethylenediamine, 1,2-propanediamine and 1,3-propanediamine, and/or
one or more alkanolamines selected from the group consisting of monoethanolamine, 2-aminopropan-1-ol and 1-aminopropan-2-ol, and/or
one or more glycerol-like specialty amines selected from the group consisting of 1,2,3-triaminopropane, 1,3-diaminopropan-2-ol, 1,2-diaminopropan-3-ol, 1-aminopropanediol and 2-aminopropanediol, and/or
piperazine, and/or
one or more piperazine derivatives selected from the group consisting of 2-methylpiperazine, 2,6-dimethylpiperazine, 2,5-dimethylpiperazine, 2,5-bis(aminomethyl)piperazine, 2,6-bis(aminomethyl)piperazine, 2-aminomethyl-5-methylpiperazine and 2-aminomethyl-6-methylpiperazine.

36. The process according to claim 32, wherein the reaction effluent comprises at least one amine selected from the group consisting of 2-aminopropan-1-ol, 1,2,3-triaminopropane, 1,2-diaminopropan-3-ol, 2-aminopropanediol, 2-aminomethyl-6-methylpiperazine, 2,5-bis(aminomethyl)piperazine and 2,6-bis(aminomethyl)piperazine.

37. A synthesis unit for the production of surfactants, medicaments and crop protection compositions, stabilizers, polymers, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerators and/or emulsifiers which comprise utilizing the amine obtainable according to claim 32, wherein the amine is a monoamine selected from the group consisting of methylamine, ethylamine, isopropylamine and n-propylamine, or of a diamine selected from the group consisting of ethylenediamine, 1,2-propanediamine and 1,3-propanediamine, or of an alkanolamine selected from the group consisting of monoethanolamine, 2-aminopropan-1-ol and 1-aminopropan-2-ol, or of a glycerol-like specialty amine selected from the group consisting of 1,2,3-triaminopropane, 1,3-diaminopropan-2-ol, 1,2-diaminopropan-3-ol, 1-aminopropanediol and 2-aminopropanediol, and/or piperazine, or of a piperazine derivative selected from the group consisting of 2-methylpiperazine, 2,6-dimethylpiperazine, 2,5-dimethylpiperazine, 2,5-bis(aminomethyl)piperazine, 2,6-bis(aminomethyl)piperazine, 2-aminomethyl-5-methylpiperazine and 2-aminomethyl-6-methylpiperazine.

38. 2-Aminomethyl-6-methylpiperazine.

39. 2,5-Bis(Aminomethyl)piperazine.

40. 2,6-Bis(Aminomethyl)piperazine.
